(19) United States
(12) Patent Application Publication
(10) Pub. No.: US 2021/0189320 A1
(43) Pub. Date: Jun. 24, 2021

(54) PROCESS AND SYSTEM FOR MICROBIAL FERMENTATION

(71) Applicants: SEKAB E-TECHNOLOGY AB, ÖRNSKÖLDSVIK (SE); TERRANOL A/S, København SV (DK)

(72) Inventors: Adnan CAVKA, ARNÄSVALL (SE); Patrik WENNBERG, ÖRNSKÖLDSVIK (SE); Thomas Hvid ANDERSEN, FREDERIKSBERG (DK); Jan Dines KNUDSEN, Malmö (SE); Rønnow BIRGITTE, København K (DK); Sibbesen OLE, Bagsværd (DK)

(21) Appl. No.: 17/050,549

(22) PCT Filed: Apr. 26, 2019
(86) PCT No.: PCT/EP2019/060706

§ 371 Date: Oct. 26, 2020

(30) Foreign Application Priority Data
Apr. 27, 2018 (EP) 18169907.5

(51) Int. Cl. C12M 1/34; C12P 7/06; C12M 1/00

(52) U.S. Cl. CPC C12M 41/32; C12M 21/12; C12P 7/06

(57) ABSTRACT

A method and system for control of a microbial fermentation process involving co-fermentation of sugars from lignocellulosic biomass to fermentation products by means of fermentation microorganisms is provided. A residual sugar indicator parameter RSI is measured, which parameter directly or indirectly indicates the concentration of residual sugars, during fermentation in the fermentation vessel (31). The amount of fermentation media added to the fermentation vessel is automatically adapted in a predetermined manner in response to the residual sugar indicator parameter, so as to achieve efficient co-fermentation of sugars to fermentation products.

### TECHNICAL FIELD

The present invention generally relates to microbial fermentation and in particular to a method and system for control of a microbial fermentation process involving co-fermentation of sugars from lignocellulosic biomass to fermentation products.

### BACKGROUND

Global warming, petroleum depletion and energy security have been the main driving forces for the development of renewable fuels that can replace the petroleum-derived fuels, such as gasoline and diesel. Microbially produced ethanol (often referred to as bioethanol) is currently the most commonly used renewable automobile fuel. It is largely produced by fermentation of sugars derived from cellulosic biomass, such as sugar- or starch-containing feedstocks, e.g. cane sugar, corn and wheat. However, the supply of these crops is relatively limited, and many of them can be considered as a human food resource. Cellulosic biomass comprising lignin (lignocellulosic biomass, also referred to simply as lignocellulose), on the other hand, is a more abundant and less expensive raw material, often considered as waste, with the potential to give a high net energy gain.

Lignocellulose is composed of cellulose, hemicellulose and lignin. Cellulose is composed of polysaccharide chains of several hundred to over ten thousand linked glucose units, whereas hemicellulose is a polysaccharide composed of xylose, other pentose sugars and various hexose sugars. In lignocellulose, cellulose and hemicellulose are tightly associated to lignin, a polyphenolic compound that ties the cellulose and hemicellulose polymers together, thus providing the lignocellulose with rigidity and mechanical strength.

In the production of ethanol from lignocellulosic materials, various pretreatment and hydrolysis steps are typically used to degrade the cellulose and hemicellulose polysaccharides in the lignocellulose to fermentable saccharides. The fermentable saccharides are then converted to ethanol by fermentation with microorganisms such as yeast or bacteria, and the ethanol is recovered by means of distillation. The yeast Saccharomyces cerevisiae, for example, metabolizes hexose sugars and is a microorganism suitable for industrial processes for cellulosic bioethanol production.

Bioethanol production by fermentation of hydrolyzed residual lignocellulosic biomass has the potential of up to 85% reduction of CO₂-emissions as compared to gasoline. However, in order to be more commercially interesting, there are still improvements to be made, including improvements of the microbial fermentation stage. Increased yield and cost reductions are continuously strived for.

### SUMMARY

A general object of the present invention is to provide an improved microbial fermentation process. A specific object is to provide an improved microbial fermentation process suitable for fermentation of lignocellulosic biomass materials to fermentation products, such as ethanol. Another object is to provide a more efficient microbial fermentation process resulting in yield improvements and/or cost reductions.

These objects are achieved in accordance with the attached claims.

Briefly, efficient microbial co-fermentation of cellulose and hemicellulose derived sugars originating from lignocellulosic biomass is achieved by automatic feed control wherein a continuous (or semi-continuous) flow of sugars into the fermentation vessel is adapted in response to a residual sugar indicator indicating the concentration of residual sugars in the fermentation vessel. The rate of change of the residual sugar indicator is determined and used for controlling the feed of sugars so as to achieve and maintain optimum/maximum fermentation rate. In this way, the present invention provides for optimized fermentation conditions and enables a favorable steady state condition to be reached in the fermentation. The steady state condition is associated with in situ propagation of microorganisms, which surprisingly has proved to render additional inoculum unnecessary in, for example, repeated fed-batch fermentation controlled in accordance with the invention. Preferably, the optimum/maximum fermentation rate is used to detect and maintain the steady state condition.

More specifically, a method for control of a microbial fermentation process involving co-fermentation of sugars from lignocellulosic biomass to fermentation products by means of fermentation microorganisms is provided. The method comprises continuous or semi-continuous addition, to a fermentation vessel, of a fermentation media comprising at least two different sugars; providing an initial active population of the fermentation microorganisms into the fermentation vessel; online measuring of a residual sugar indicator parameter RSI, which parameter directly or indirectly indicates the concentration of residual sugars, during fermentation in the fermentation vessel; determining an RSI setpoint based on a rate of change of the measured residual sugar indicator parameter, such that the RSI setpoint corresponds to a maximum rate of change; and automatically adapting the amount of sugar added to the fermentation vessel in a predetermined manner in response to the measured residual sugar indicator parameter RSI and the RSI setpoint, so as to achieve and maintain the RSI setpoint, whereby efficient co-fermentation of sugars to fermentation products is obtained.

According to an advantageous embodiment, the RSI setpoint corresponds to a steady state condition in the fermentation vessel and the method further comprises in situ growing of the fermentation microorganisms in the fermentation vessel by means of the sugars added to the fermentation vessel, so as to maintain a steady-state population of fermentation microorganisms in the fermentation vessel, whereby further addition of microorganisms is not required.

Advantages associated with the present invention, are inter alia:
more efficient co-fermentation
utilization of a higher proportion of the sugars available
yield improvement
increased volumetric productivity (cost reduction)
reduced operational costs, e.g. yeast cost reduction due to in situ propagation
cost reduction in yeast fermentation for commercial production of cellulosic ethanol

The RSI setpoint may, according to one embodiment, comprise a target interval of the RSI or a target interval of the RSI rate of change, and the step of automatically adapting in turn comprises the steps of: comparing measured, and preferably processed, values of the residual sugar indicator parameter to the RSI setpoint; and adjusting the amount of sugar added to the fermentation vessel in response to the comparison, so as to reach and stay within the RSI setpoint.

The fermentation process may, according to one embodiment, be a fed-batch fermentation process comprising at least one batch phase, feed phase and end phase, respectively, and having continuous addition of fermentation media during the at least one feed phase.

The fed-batch fermentation process may, according to one embodiment, be a repeated fed-batch fermentation process with continuous addition of fermentation media during at least two feed phases.

The respective feed phase or feed phases of the fermentation process may, according to one embodiment, be extended so as to comprise a substantial portion of the fed-batch cycle.

The fermentation process may, according to one embodiment, be a fed-batch fermentation process wherein the step of determining an RSI setpoint is based on initial RSI measurements during the batch phase, and the step of automatically adapting comprises automatically adapting, during the feed phase, the amount of sugar added to the fermentation vessel in a predetermined manner in response to further RSI measurements during the feed phase and said RSI setpoint, so as to achieve and maintain said RSI setpoint. The step of providing an initial active population is preferably preceded by partially filling the fermentation vessel with fermentation media.

The fermentation process may, according to one embodiment, be a fed-batch fermentation process with continuous addition of fermentation media but substantially no addition of microorganisms during the feed phase(s).

The fermentation process may, according to one embodiment, comprise a step of secondary fermentation, in at least one secondary fermentation vessel arranged downstream of the fermentation vessel, to further increase the co-fermentation of sugars to fermentation products.

The fermentation process may, according to one embodiment, be a fed-batch fermentation process wherein the secondary fermentation comprises directing an overflow of the fermentation vessel into at least one secondary fermentation vessel to finish the fermentation of the residual sugars.

The fermentation process may, according to one embodiment, have a measuring step which involves density measurements.

The measuring step may, according to one embodiment, involve refractive index (RI) measurements and the residual sugar indicator parameter RSI may comprise a refractive index (RI) parameter.

The measuring step may, according to one embodiment, involve a combination of measurements, i.e. at least two different measurements, selected from the group of: optical measurements within UV, visual or IR wavelengths; measurements of carbon dioxide (CO₂) generation; and direct measurements of sugar concentration, preferably using chromatography, sugar assay kits, or glucometers.

The fermentation process may, according to one embodiment, comprise addition, to the fermentation vessel, of at least two separate sugar streams associated with the respective different sugars; and individual adjustment of the respective at least two separate sugar streams associated with the respective different sugars.

The fermentation process may, according to one embodiment, comprise recirculation of fermentation microorganisms from a position downstream of the fermentation vessel and back into the fermentation vessel.

According to another aspect of the invention a system for control of a microbial fermentation process is provided.

More specifically, a system for control of a microbial fermentation process involving co-fermentation, in a fermentation vessel, of sugars from lignocellulosic biomass to fermentation products by means of fermentation microorganisms is provided. The system comprises means for continuous or semi-continuous addition, to the fermentation vessel, of a fermentation media comprising at least two different sugars; measuring means for online measuring of a residual sugar indicator parameter RSI, which parameter directly or indirectly indicates the concentration of residual sugars, during fermentation in the fermentation vessel; means for determining and setting an RSI setpoint based on a rate of change of the measured residual sugar indicator parameter, such that the RSI setpoint corresponds to a maximum rate of change; and control means for automatically adapting the amount of sugar added to the fermentation vessel in a predetermined manner in response to the measured residual sugar indicator parameter RSI and the RSI setpoint, so as to achieve and maintain the RSI setpoint, whereby efficient co-fermentation of sugars to fermentation products is obtained.

According to a preferred embodiment, the system further comprises means for in situ growing of the fermentation microorganisms in the fermentation vessel by means of the sugars added to the fermentation vessel, so as to maintain a steady-state population of fermentation microorganisms in the fermentation vessel, the RSI setpoint corresponding to the steady state condition in the fermentation vessel.

According to one embodiment, the RSI setpoint comprises a target interval of the RSI or a target interval of the RSI rate of change, and the control means for automatically adapting in turn comprises: means for comparing measured, and preferably processed, values of the residual sugar indicator parameter to the RSI setpoint; and means for adjusting the amount of sugar added to the fermentation vessel in response to the comparison, so as to reach and stay within the RSI setpoint.

According to one embodiment, the system is a fed-batch fermentation system arranged for fed-batch fermentation in at least one batch phase, feed phase and end phase, respectively, with continuous addition of fermentation media during the at least one feed phase.

According to one embodiment, the system comprises a secondary fermentation unit arranged downstream of the fermentation vessel, for further increased co-fermentation of sugars to fermentation products, preferably in at least two secondary fermentation vessels connected in series and/or at least two secondary fermentation vessels connected in parallel.

According to one embodiment, the system comprises refractive index (RI) measurement means.

According to one embodiment, the system comprises means for separate addition, to the fermentation vessel, of at least two separate sugar streams associated with the respective different sugars; and means for individual adjustment of the respective at least two separate sugar streams associated with the respective different sugars.

According to one embodiment, the system comprises means for recirculation of fermentation microorganisms from a position downstream of the fermentation vessel and back into the fermentation vessel.

According to yet another aspect of the invention a system for producing fermentation products, preferably including ethanol, from lignocellulosic biomass is provided.

### BRIEF DESCRIPTION OF THE DRAWINGS

The invention, together with further objects and advantages thereof, is best understood by reference to the following description and the accompanying drawings, in which:

FIG. 1 is a schematic view of a system for producing ethanol from lignocellulosic biomass according to an exemplary embodiment of the present invention;

FIG. 2 is a schematic view of a fermentation control system according to an exemplary embodiment of the present invention;

FIG. 3 is a flow chart of an exemplary embodiment of the method for fermentation control according to the present invention;

FIG. 4 is a flow chart of another exemplary embodiment of the method for fermentation control according to the present invention;

FIG. 5 is a diagram illustrating refractive index (RI) and residual sugars during fermentation with automatic feed control according to an exemplary embodiment of the present invention;

FIG. 6 is a diagram illustrating fermentation with automatic feed control according to another exemplary embodiment of the present invention;

FIG. 7 is a diagram illustrating fermentation with automatic feed control according to another exemplary embodiment of the present invention with secondary fermentation; and

FIGS. 8 A and B are schematic drawings illustrating secondary fermentation configuration with serial and parallel mode according to exemplary embodiments of the present invention.

### DETAILED DESCRIPTION

Throughout the drawings the same reference numbers are used for similar or corresponding elements.

The term microbial fermentation herein refers to fermentation by microorganisms. The term co-fermentation herein refers to the simultaneous fermentation of two (or more) different sugars in the same fermentation reactor/process step.

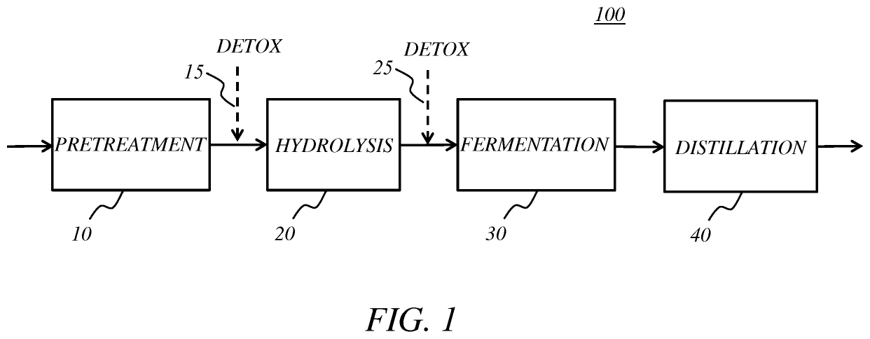

FIG. 1 is a schematic view of a system 100 for producing ethanol from lignocellulosic biomass according to an exemplary embodiment of the present invention. In the illustrated example, input raw material or source material is processed in a pretreatment unit 10 and a hydrolysis unit 20 before the hydrolysate enters a fermentation unit/system 30. Various pretreatment and hydrolysis units/systems known in the art can be used to degrade the polysaccharides in the lignocellulose to fermentable sugars. The pretreatment unit 10 may for instance also include an impregnation unit.

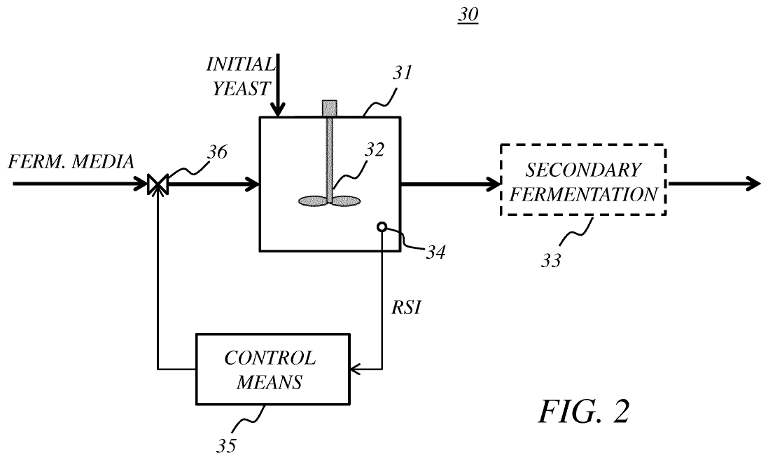

The fermentation unit/system 30 comprises one or more fermentation vessels/reactors (31 in FIG. 2) in which two or more fermentable sugars are converted to ethanol by fermentation with microorganisms. The ethanol is recovered by means of distillation in a distillation unit/system 40 connected to the fermentation unit 30.

Preferred embodiments of the present invention have separate hydrolysis and fermentation units (SHF) 20, 30, which facilitates the feed control. However, under certain circumstances it may also be possible to use the inventive feed control in systems with simultaneous saccharification and fermentation (SSF), i.e. hydrolysis and fermentation in the same fermentation reactor.

In FIG. 1 and other herein described embodiments, the primary fermentation product is exemplified as ethanol. However, it should be understood that the present invention is also applicable to processes resulting in other fermentation products, such as other alcohols, organic acids and fatty acids. According to one advantageous embodiment the fermentation product is lactic acid.

The source material in the process and system of the present invention may be any lignocellulosic biomass, including softwood and hardwood. Fermentation media comprising lignocellulosic biomass or thereof derived sugars is input to the fermentation system, where sugars from the lignocellulosic biomass input material are co-fermented. The fermentation media may often be a hydrolysate from a preceding hydrolysis stage.

As illustrated by dashed arrows in FIG. 1, there may be an optional detoxification 15 between the pretreatment and hydrolysis units 10, 20 and/or an optional detoxification 25 between the hydrolysis and fermentation units 20, 30. Using such a detoxified hydrolysate as fermentation media has advantages in connection with the present invention, as will be described in the following.

FIG. 2 is a schematic view of a system 30 for controlled fermentation according to an exemplary embodiment of the present invention. The system 30 comprises a fermentation vessel (i.e. fermentation reactor) 31 which receives a fermentation media, such as a hydrolysate, comprising at least two different sugars. In the fermentation vessel 31, co-fermentation of different sugars is achieved by means of suitable conventional co-fermentation microorganisms, such as Saccharomyces cerevisiae fermentation yeasts suitable for ethanol production from lignocellulosic biomass and modified by metabolic engineering. Yeast strains commercially available from the company Terranol A/S, including strain V1, cv-40 and cv-110, may for example be used. The fermentation vessel 31 has agitation means 32, e.g. a conventional rotor with associated motor. The agitation means 32 is preferably arranged so as to achieve mixing throughout substantially the entire fermentation vessel 31, to facilitate the contact between the microorganisms and fermentation media.

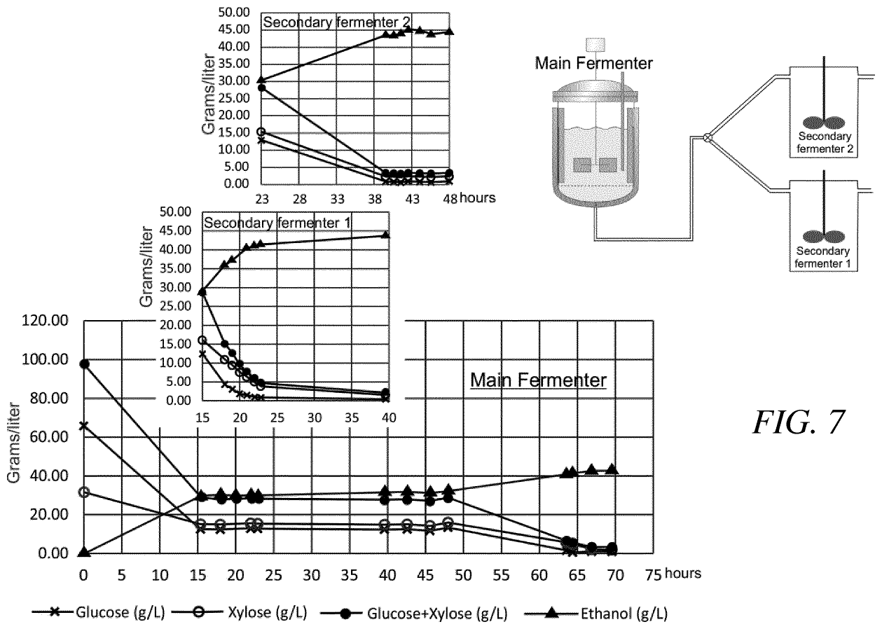

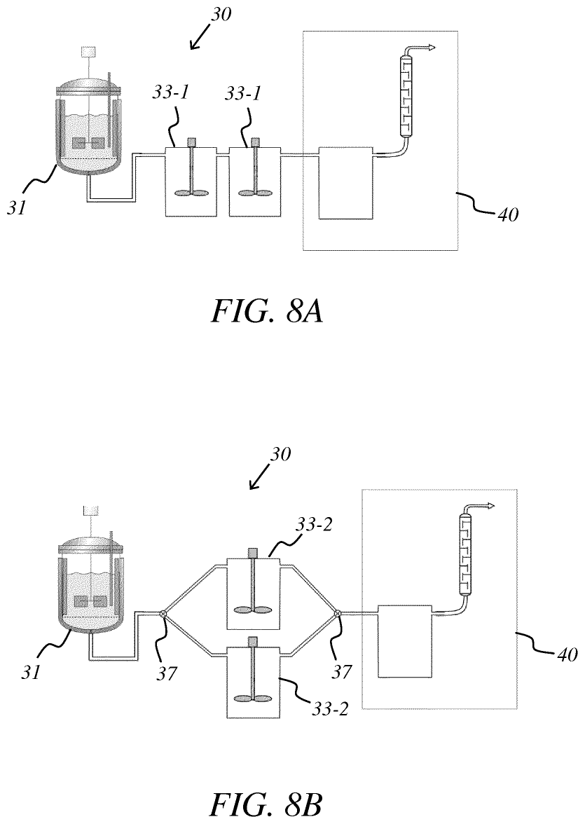

Downstream the fermentation vessel 31, there is an optional secondary fermentation stage 33 for further fermentation of sugars to fermentation products. (Preferred embodiments of the secondary fermentation are illustrated in FIGS. 7 and 8 and further described below.)

The fermentation control system 30 further comprises means 34, 35 for measuring of a residual sugar indicator parameter RSI, which directly or indirectly indicates the concentration of residual sugars in the fermentation vessel 31. The measuring means typically includes one or more sensors 34 arranged in the reactor (FIG. 2) or in a loop outside the reactor (for example using a recirculation pump). There may also be embodiments with sensors in the reactor outflow (of output liquid fermentation products or output CO₂). Sensors can in addition also be arranged in the reactor inflow, providing RSI signals used in combination with RSI measured in the outflow.

Both inline and online RSI measurements are thus possible.

The measured RSI signal is received by control means 35, which may e.g. be a computer control means designed to automatically adapt the amount of sugar added to the fermentation vessel 31 in a predetermined manner in response to the measured RSI signal. In the example of FIG. 2, the control means 35 communicates with a valve 36 so as to achieve the variable fermentation media stream input to the fermentation vessel 31, but the skilled person understands that other equipment and process designs are possible.

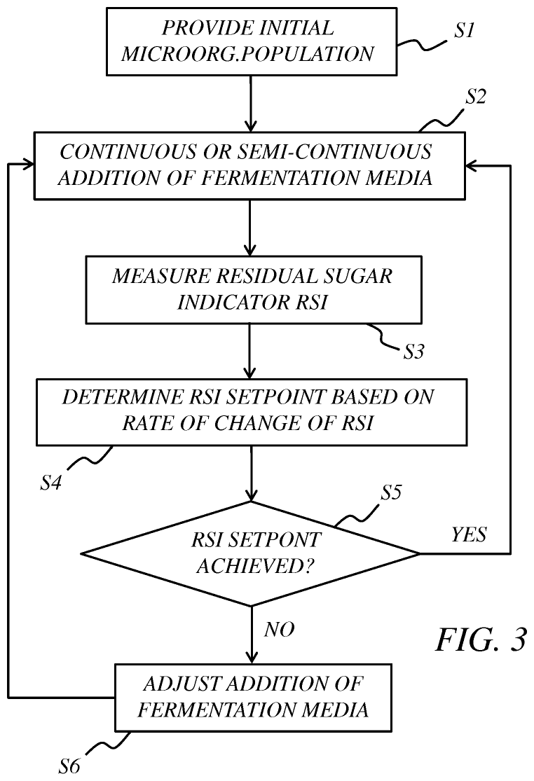

FIG. 3 is a flow chart of an exemplary embodiment of the method for control of a microbial fermentation process involving co-fermentation of sugars from lignocellulosic biomass according to the present invention. In step S1, an initial population of fermentation microorganisms is provided, by addition into the fermentation vessel. A fermentation media comprising at least two different sugars is continuously added to the fermentation vessel (step S2). The addition of fermentation media is performed as a continuous feed for a substantial time, in continuous or semi-continuous operation.

At the point in time when the initial microorganism population is added into the fermentation vessel, the fermentation vessel would typically be containing, or receiving, an amount of fermentation media.

In step S3, a residual sugar indicator parameter RSI, which directly or indirectly indicates the concentration of residual sugars, during fermentation in the fermentation vessel, is measured. The RSI may be a density indicator, for example obtained using optical measurements or refractive index (RI) measurements. The RSI could also be derived from measurements and calculations of carbon dioxide (CO₂) generation, or obtained by direct measurements of sugar concentration.

An RSI setpoint is in step S4 determined based on a rate of change of the measured values of the residual sugar indicator parameter RSI, such that the RSI setpoint corresponds to a maximum or optimum rate of change. The RSI setpoint is a target parameter (also referred to as desired parameter or reference parameter). It may be a target value or an interval corresponding to desirable or optimum process conditions. The RSI setpoint may be fixed or variable. The determining of the RSI setpoint may involve calculations and/or online following of the registered RSI signal during the fermentation process. In a preferred embodiment, the RSI setpoint corresponds to a steady state condition in the fermentation vessel.

According to the invention, the amount of sugar added to the fermentation vessel is automatically adapted in a predetermined manner in response to the measured residual sugar indicator parameter RSI and the RSI setpoint, so as to achieve and maintain the RSI setpoint, whereby efficient co-fermentation of sugars to fermentation products is obtained.

Step S5 asks if the RSI setpoint is achieved. This could mean comparing the measured, and possibly processed, RSI values (registered actual RSI) to the RSI setpoint. It could for example also mean comparing calculated derivative values of the registered RSI to a RSI setpoint defined as the maximum or optimum rate of change of RSI. If the desired interval is not met, the amount of sugar added to the fermentation vessel is adjusted in response to the comparison, so as to achieve (or come closer to) a desired fermentation rate (step S6). If, on the other hand, the RSI setpoint is reached, i.e. the rate of change is within the desired interval, the fermentation media addition can continue without adjustment and the process returns to step S2.

The present invention refers to fermentation with continuous feed of fermentation media for the whole or part of the process running time (i.e. for at least a portion of the process running time). In other words, the addition of the fermentation media to the fermentation vessel according to the present invention may be continuous or semi-continuous.

The operation of the process may be continuous with continuous addition of fermentation media during substantially the whole process running time.

The operation of the process may for example be prolonged into a continuous phase after completed filling of the fermentation vessel by continuous controlled feed or continuous addition of fermentation media.

The operation of the process is preferably fed-batch or repeated fed-batch, with continuous addition of fermentation media during the at least one feed phase.

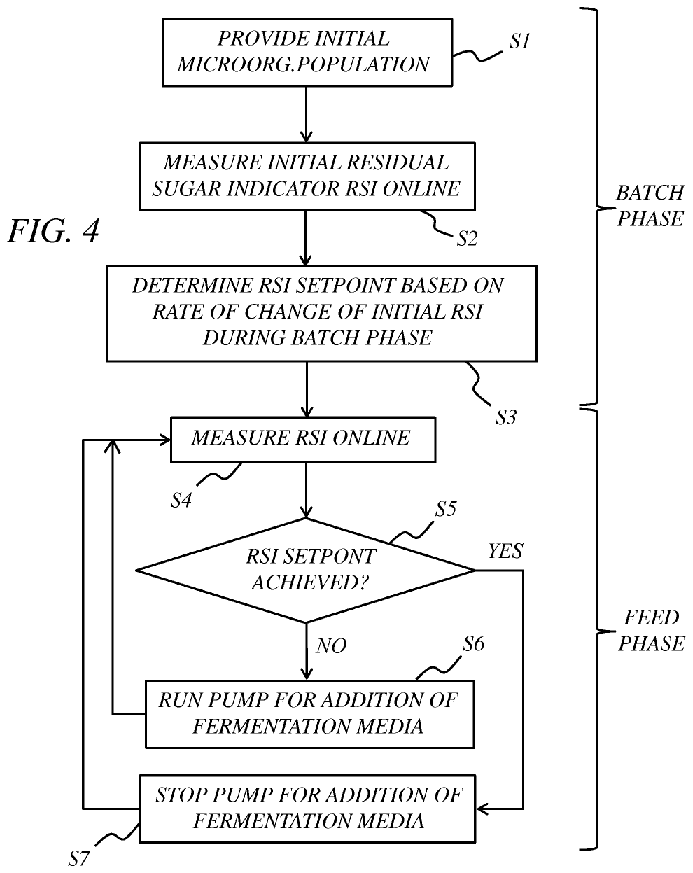

FIG. 4 is a flow chart illustrating an exemplary embodiment of the present invention, with automatic control of a fed-batch fermentation process. In step S1, an initial population of fermentation microorganisms is added into a fermentation vessel containing an amount of lignocellulosic biomass hydrolysate comprising at least two different sugars.

In step S2, a residual sugar indicator parameter RSI, which directly or indirectly indicates the concentration of residual sugars, is measured at the onset of the fermentation in the fermentation vessel. The RSI may be a density indicator, for example obtained using optical measurements or refractive index (RI) measurements. The RSI could also be derived from measurements and calculations of carbon dioxide (CO₂) generation, or obtained by direct measurement of sugar concentration.

In step S3 the RSI setpoint is determined by calculation based on initial RSI measurements or by another determination based on online measured RSI during the batch phase of the fermentation. In step S4, the continuous online measurement of the RSI parameter proceeds after the RSI setpoint has been inserted or set. In step S5, the measured RSI value is compared with the RSI setpoint. Addition of a fermentation media comprising at least two different sugars is set in run state (S6) or in stop state (S7) according to the result in S5. The measurement in S4, the decision in S5 and the running or stopping of the pump is repeated with preset fixed intervals that may be measured in seconds, minutes or hours. Overflow from the fermentation vessel is preferably collected in a secondary fermentation stage to complete the fermentation.

In accordance with the present invention, a residual sugar indicator parameter RSI is measured and used to control the fermentation. RSI directly or indirectly indicates the concentration of residual sugars, i.e. the remaining sugars (individual or total) during the microbial fermentation in the fermentation vessel. RSI is used as a control parameter in the automatic feed control.

In some embodiments, RSI is a density indicator, obtained from online density measurements of the content of the fermentation vessel. Refractive index (RI) measurements may for example be used. Optical measurements within UV, visual or IR wavelengths may also be used.

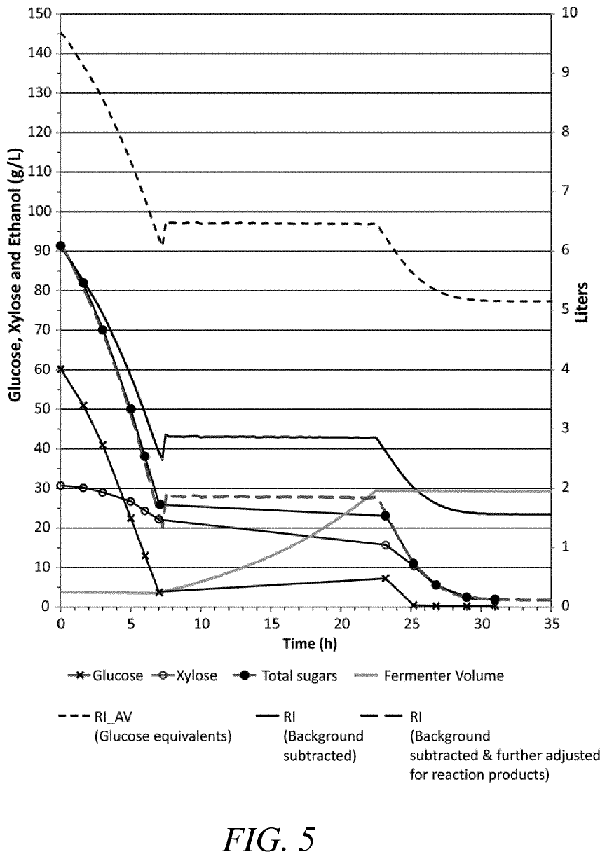

A preferred embodiment with RI (refractive index) controlled feeding is illustrated by the example diagram of FIG. 5, which relates to fed-batch fermentation with online RI monitoring and control. Residual sugars (glucose, xylose, and total sugars) determined by HPLC and RI (in glucose equivalents) are shown. The comparatively simple RI measurements have proven to reflect the sugar concentrations from HPLC surprisingly well. FIG. 5 implies that RI in particular is very useful as residual sugar indicator RSI for the automatic feed control in accordance with the present invention.

With processing of the output RI signal (adjusting for background and relating the signal output with for instance HPLC data) it can be directly correlated to that of residual sugars. The inventors have shown that it is possible to calculate residual fermentable sugars from online RI during the fermentation through this method with high accuracy.

Thus, online RI measurements can be used either unprocessed or processed to estimate/calculate residual sugar content, for fast and efficient co-fermentation control in accordance with preferred embodiments of the present invention.

RI is preferably measured online in the fermenter, or in a looped tube circulating fermentation broth in the fermenter, or in the outlet tube. RI is preferably measured with a suitable refractometer.

Process conditions of the FIG. 5 example: Lignocellulosic (wheat straw) hydrolysate with solids. Yeast strain cV-110 (Terranol), 0.5 g/L total pitch (4 g/L initially in batch), pH 5.2, 32° C.

Indirect residual sugar indicators RSI may also be used, for example obtained from carbon dioxide (CO₂) measurements. CO₂ can then be measured as such with CO₂ gas sensors, or, alternatively, be indirectly measured preferably via gas pressure P or via the degree of opening of the CO₂ exhaust valve.

The use of CO₂ generation as the RSI parameter would typically require an online calculation to transform the total generated CO₂ amount into an amount of fermented sugar and the corresponding change of concentration of sugars in the fermentation vessel.

It would even be possible to use the ethanol output from the fermentation process as indirect residual sugar indicator RSI. The sugar content is then derived from the well-known relationship between sugar, CO2 and ethanol.

There are also embodiments of the invention which use direct measurements of sugar concentration, preferably using chromatography, sugar assay kits, or glucometers.

For increased control accuracy and process efficiency, there are also embodiments of the invention which use a combination of measurements, for example density and CO₂ measurements, or density, HPLC and CO₂ measurements.

The registered signal of the residual sugar indicator parameter RSI is generally processed using a mathematical function, such as a moving average and/or derivate of the residual sugar indicator.

The desired parameter value/interval, RSI setpoint, may be changed during operation, manually or automatically. It may also be set to be dependent of, i.e. as a mathematical function of, one or more other parameters.

By means of the fermentation control according to the present invention, efficient co-fermentation of sugars from lignocellulosic biomass can be achieved. Adapting the feed of fermentation media, e.g. hydrolysate, into the fermentation vessel in response to the registered residual sugar indicator RSI in the manner proposed by the invention leads to a more efficient use of the sugars available and utilizing of a higher proportion of the sugars available.

This means yield improvements by increased fermentation efficiency and also cost reductions due to increased volumetric productivity. The invention also enables reduced operational costs (including reduced costs for yeast).

According to the invention the feed of fermentation media is adapted so as to optimize the fermentation rate. This is based on the insight that an optimum fermentation rate corresponds to a steady state condition of the microbial fermentation. In the steady state condition, RSI may thus be maintained at substantially the same level despite continuous addition of fermentation media and no addition of microorganisms.

The inventors have unexpectedly found that, after an initial population of microorganisms, typically yeast, is provided, additional inoculum is not required during fermentation controlled in accordance with the present invention. The hypothesis was that, due to dilution of the yeast, additional inoculum (external supply) would be needed between phases in repeated fed-batch operation, for example. However, it was surprisingly discovered that the yeast concentration is maintained. The fermentation vessel can be emptied until only about 20-25% of the volume remains, and still no refill of yeast is needed since the yeast is, by means of the at least partially continuous stream of sugar added, growing with increased volume in the fermentation vessel.

Basically, the microorganisms (e.g. yeast) will, provided that they have sufficient sugar, grow until an optimum/maximum rate of fermentation is achieved. This optimum fermentation rate is detected via the RSI measurements in the feed controlled system according to the present invention. At the optimum fermentation rate a steady-state condition is achieved by in situ propagation.

The process of the invention preferably comprises achieving in-situ propagation of fermentation microorganisms resulting in a steady-state population so that additional inoculum is not required during fermentation. This means that the added amount of sugar is adapted so as to achieve and maintain the optimum fermentation rate and hence the steady-state population of microorganisms.

The desirable steady state condition would in accordance with embodiments of the invention be detected via determining the optimum, i.e. typically the maximum, fermentation rate of the process from RSI measurements. The steady state condition is achieved at an RSI that is the same at least for a substantial period of time.

The amount of sugars available would in general be decisive for the yeast growth (in situ propagation) and the desirable steady-state condition could also be expressed as an optimum yeast to sugar ratio, e.g. determined from online measurements of sugar and yeast concentration.

The system will automatically reach a new steady state in response to changed conditions. If, for example, the ratio sugar:yeast is altered by manually lowering or increasing the sugar concentration, the yeast will adjust, by either growing further to a new steady state or, in case of shortage of sugar in relation to yeast, some starvation in the population will occur, reaching a new steady state with lower yeast concentration.

FIGS. 5, 6 and 7 are diagrams illustrating fed-batch fermentation with automatic feed control according to exemplary embodiments of the present invention.

FIG. 5 illustrates one phase fed-batch fermentation of wheat straw derived lignocellulosic material with automatic feed control according to the invention. The volume of fermentation media in the fermentation vessel during the respective batch, feed and end phases is shown. (The volume increase indirectly illustrates the addition of fermentation media.) Sugar content (glucose and xylose, in this example) is shown. RI (refractive index) is used as residual sugar indicator, i.e. the feed control is performed using online RI measurements as input signal.

It is clear from FIG. 5 that RI is very suitable for indicating residual sugars during fermentation with automatic feed control according to the present invention.

The one phase fed-batch process of FIG. 5 has a comparatively short feed phase (about 15 h, or about 50% of the fed-batch cycle). As shown by FIG. 5, low content of residual sugars, i.e. efficient fermentation, is still achieved with one phase fed-batch fermentation controlled in accordance with the invention. A high ethanol yield (>90%) was obtained.

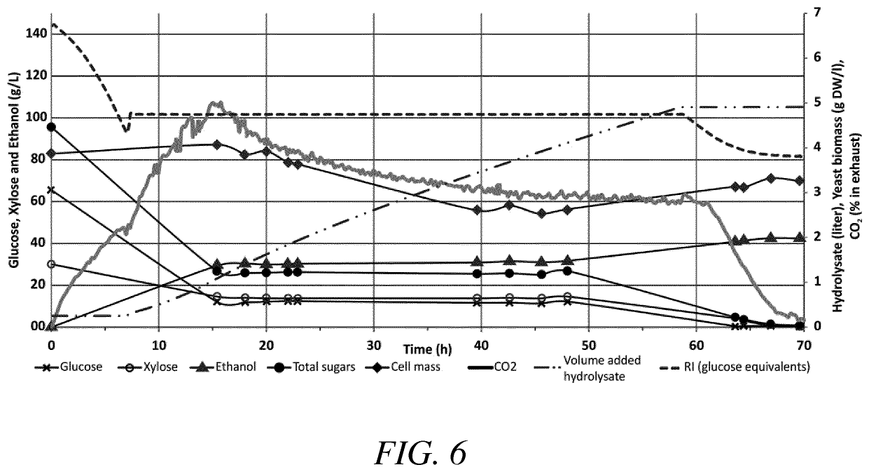

FIG. 6 illustrates fed-batch fermentation of lignocellulosic material with automatic feed control according to another embodiment of the invention, using an extended (also referred to as prolonged or continued) feed phase.

The lignocellulosic material used as fermentation media is in this example wheat straw hydrolysate. The wheat straw hydrolysate substrate was supplied with urea as nitrogen source (3 g/l) and adjusted to pH 5.5. The same hydrolysate was used for the batch phase and the feed phase.

The batch phase (25% of full fermenter filling of 1 liter) was inoculated with 3.9 gram/liter DW of yeast strain cV-110.

The volume of fermentation media in the fermentation vessel during the respective batch, feed and end phases is shown. (The volume increase indirectly illustrates the addition of fermentation media.) Sugar content (glucose and xylose, in this example) is shown, as well as the fermentation products ethanol and CO₂. RI is used as residual sugar indicator, i.e. the feed control is performed using online RI measurements as input signal.

When the RI value corresponding to the highest RI change rate was found, this was used as setpoint throughout the rest of the feed phase. The pH was maintained and controlled using dilute sulphuric acid and 20% ammonia solution, and the temperature kept at 32° C.

As illustrated by FIG. 6, the continued fed-batch fermentation process with automatic feed control of the invention results in a high ethanol yield (>90%) and low content of residual sugars. Both glucose and xylose were consumed by the fermentation microorganisms to extremely low levels.

The one phase fed-batch process of FIG. 6 has a comparatively long feed phase (about 45 h, or about 65% of the illustrated fed-batch cycle). This is advantageous inter alia due to the fact that the ethanol productivity (g ethanol per g yeast and time) is higher during the feed phase than during the batch phase and end phase.

In a process with extended feed phase(s), the respective feed phase or feed phases of the fermentation process would be extended a certain time beyond the point where the fermenter is full, by continuing the feeding and removing the surplus overflow of fermentation broth from the fermenter. An extended feed phase preferably has a length corresponding to at least 60%, more preferably at least 70%, of the fed-batch cycle.

As mentioned earlier in connection with FIG. 2, preferred embodiments of the present invention may also include secondary fermentation, in at least one secondary fermentation vessel 33 arranged downstream of the fermentation vessel 31, to further increase the co-fermentation of sugars to fermentation products.

In the example illustrated by FIG. 7, the fermentation process of FIG. 6, in a main fermenter, is combined with a secondary fermentation process in secondary fermenters.

Upon continued fed-batch fermentation of lignocellulosic material with automatic feed control according to FIG. 6, the overflow of the main fermenter was directed into two parallel connected secondary fermenters. The feeding was continued for several additional fermenter volumes and the overflow directed into a waste container, which showed that at the late phase at 48 hours and forwardly a steady state of CO₂ generation is obtained.

The secondary tanks were stirred and thermostated at 30° C., without any other controls or adjustments. If the secondary tanks are sufficiently insulated, only stirring is needed.

Feeding to the main fermenter was stopped after feeding of 5 fermenter volumes (5 liter, at 58 hours) and the fermenter stirring remained until the fermentation was finished after approximately 68 hours.

Continued (i.e. extended) fed-batch, as in FIGS. 6 and 7, results in a higher ethanol yield as higher yield is obtained in the feed phase and end phase compared to the batch phase. A further advantage is that the prolonged feed phase when used with the feed control in accordance with the present invention, results in increased cost-efficiency due to a more efficient use of the microorganisms (typically yeast).

Due to in situ propagation the same initial population of microorganisms can be used for an extended feed phase. This results in reduced yeast expenditure, and consequently in lower operational costs. FIG. 6 clearly illustrates that the fermentation efficiency can be maintained in a steady state with no yeast refill.

FIG. 7 shows that secondary fermentation in at least two secondary fermentation vessels provides for efficient handling of the prolonged feed phase and leads to improved fermentation results. High ethanol yield (>90%) and extremely low levels of residual sugars are achieved.

Similar advantageous effects as with the prolonged feed phase have been observed by the inventors during experiments with repeated fed-batch operation with continuous addition of fermentation media during at least two feed phases and wherein the fermentation vessel is only partially emptied between the respective feed phases.

Repeated fed-batch (diagram not shown) results in a high ethanol yield and low content of residual sugars. A further advantage is that two or more consecutive feed phases in the inventive fermentation with feed control result in increased cost-efficiency due to a more efficient use of the microorganisms (typically yeast).

Due to in situ propagation the same initial population of microorganisms can be used again and again. Two or more feed phases without emptying the fermentation vessel in between, result in reduced yeast expenditure, and consequently in lower operational costs. In embodiments of the present invention using repeated feed phase operation, the fermentation efficiency can be maintained with no yeast refill.

The favorable extended, i.e. comparatively long, feed phase can be useful in connection with one phase fed-batch or repeated fed-batch fermentation. As mentioned, such an extended feed phase may be combined with secondary fermentation for efficient handling of the end phase and improved fermentation results.

Secondary fermentation will now be described more in detail with reference to FIGS. 7 and 8.

The secondary fermentation is a post-fermentation, typically without further addition of fresh fermentation media or fermentation microorganisms. Sugar remaining after the primary fermentation can thus be consumed in the secondary fermentation. The operation of the secondary fermentation may be batch, fed-batch or continuous.

The secondary fermentation vessel(s) 33 preferably has agitation means, e.g. a conventional rotor with associated motor. The agitation means is preferably arranged so as to achieve mixing throughout substantially the entire fermentation vessel to facilitate the contact between the microorganisms and fermentation media.

Preferably, there are at least two secondary fermentation vessels, connected in series or in parallel. Principles of serial and parallel configurations are schematically illustrated in FIGS. 8A and 8B.

FIG. 8A shows a fermentation system 30 with two serially connected secondary fermentation vessels 33-1, arranged downstream of a main fermentation vessel 31 with feed control and upstream of a distillation facility 40. In order to achieve a most efficient fermentation process, the secondary fermentation system is arranged such that there is minimum intermixing between the first and the second secondary fermentation vessel 33-1. One vessel is preferably completely emptied before the filling of the other vessel begins, and so on. In this way, there is time to finish fermentation and thus to reach extremely low levels of residual sugars.

FIG. 8B shows a fermentation system 30 with two parallel connected secondary fermentation vessels 33-2, arranged downstream of a main fermentation vessel 31 with feed control and upstream of a distillation facility 40. In order to achieve a most efficient fermentation process, the secondary fermentation system is arranged such that there is minimum intermixing between the first and the second secondary fermentation vessel 33-2. Flow switching means such as conventional valves 37 are arranged so as to switch back and forth between the two vessels 33-2. This enables fermentation to be completed with extremely low levels of residual sugars. FIG. 8B corresponds to the secondary fermentation set-up used in the process of the example diagrams in FIG. 7.

The number of secondary fermentation vessels 33-1, 33-2 may vary within the scope of the invention and various combinations of serial and parallel vessels are also possible. However, it is preferred to have at least two secondary fermentation vessels, as in FIGS. 8A and 8B, and these may with advantage be arranged for interrupted or intermittent flow so as to minimize the mixing between the vessels.

A set up with more than one connected fermentation vessel 33-1, 33-2 in the secondary fermentation is especially useful in combination with fed-batch fermentation or repeated fed-batch fermentation with extended, i.e. comparatively long, feed phase. An extended feed phase may for example be between 24 and 72 hours, its length depending e.g. on raw material, sugar combination, and inhibitor concentration. The connected tanks then take care of the end phase so that the downstream piping, e.g. to the distillation unit, can be of manageable length.

As illustrated by the examples herein (including but not limited to FIG. 5-8), the present invention results in improved fermentation of different sugars, i.e. improved co-fermentation of sugars, from lignocellulosic biomass. The fermentation media input to the fermentation vessel 31 comprises at least two different sugars from lignocellulosic biomass. These are preferably selected from the group of: glucose, mannose, xylose, arabinose, galactose, sucrose and fructose. It should be understood that the primary target molecule can be, for instance, xylose or mannose instead of glucose.

According to another advantageous embodiment of the present invention, the addition of fermentation media involves addition of at least two separate sugar streams associated with different sugars. The sugar streams may with advantage be individually adjusted in response to the registered RSI. This further improves the fermentation efficiency.

Separate sugar streams result in improved yield compared to utilizing an initial batch fermentation. As sugar streams are separate this also allows for more flexible fermentation control, i.e. sugars can be added at any rate to create optimal ratios between different sugar streams. (The optimal ratio for glucose:xylose, for example, may not be the same as for glucose: mannose or other combinations.)

Individually adjusted separate streams may also be beneficial for the in situ yeast propagation, since it enables addition of more glucose in relation to the secondary sugar(s) in order to achieve increased biomass formation (microorganism growth), which is sometimes desirable. An excess of glucose, which the microorganisms use for growth, would for instance be favorable if the population over time shows decreased efficiency or performance.

According to another advantageous embodiment of the present invention, the fermentation media added to the fermentation vessel 31 comprises a detoxified hydrolysate from preceding detoxification and hydrolysis steps. The detoxification is a treatment/conditioning performed before the fermentation in order to alleviate inhibition problems caused by substances (byproducts) formed during the pretreatment. The detoxification 15, 25 may, as illustrated using dashed arrows in FIG. 1, either be performed before or after the hydrolysis. A detoxification treatment before the hydrolysis would often be preferred when enzymatic hydrolysis is used, since the same byproducts would typically be problematic for both enzymes and fermentation microorganisms. Examples of detoxification methods which may be used include detoxification with reducing agents, overlimiting/alkali detoxification, ion exchange resins, and activated carbon.

By means of a detoxified hydrolysate, the fermentation control of the present invention becomes even more efficient and cost-effective. The feed phase (of a fed batch process) becomes shorter as compared to when the hydrolysate does not undergo detoxification. Moreover, the lag phase, when the microorganisms to adapt to the growth conditions and get started with the fermentation, is also reduced, which is a further advantage.

According to another advantageous embodiment of the present invention, there is a recirculation of yeast back into the fermentation vessel in order to further improve the fermentation efficiency and assist in eliminating the need for yeast refill in a fed-batch system. The yeast is then recirculated from a position downstream the fermentation vessel and back into the inlet or initial portion of the fermentation vessel. In systems with secondary fermentation, this downstream position may be between fermentation vessel and secondary fermentation or immediately after the secondary fermentation. A combination of fed control maintaining a steady state population and recirculation of a portion of the yeast output from the fermentation stage may be used to achieve fast and cost-efficient fermentation. Before recirculation, the yeast would generally need to be separated from other material in the liquid output from the fermentation vessel.

In the illustrated examples, strain cv-110 (available from Terranol A/S)) was used, but other microorganisms suitable for co-fermentation may of course also be used.

Although the invention has been described with reference to specific illustrated embodiments, it should be emphasized that it also covers equivalents to the disclosed features, as well as modifications and variants obvious to a man skilled in the art. Thus, the scope of the invention is only limited by the enclosed claims.

1. A method for control of a microbial fermentation process involving co-fermentation of sugars from lignocellulosic biomass to fermentation products by means of fermentation microorganisms, comprising the steps of:
continuous or semi-continuous addition, to a fermentation vessel of a fermentation media comprising at least two different sugars;
providing an initial active population of the fermentation microorganisms into the fermentation vessel;
online measuring of a residual sugar indicator parameter RSI, which parameter directly or indirectly indicates the concentration of residual sugars, during fermentation in the fermentation vessel;
determining an RSI setpoint based on a rate of change of the measured residual sugar indicator parameter, such that the RSI setpoint corresponds to a maximum rate of change; and
automatically adapting the amount of sugar added to the fermentation vessel in a predetermined manner in response to the measured residual sugar indicator parameter RSI and the RSI setpoint, so as to achieve and maintain the RSI setpoint, whereby efficient co-fermentation of sugars to fermentation products is obtained.

2. The method of claim 1, further comprising the step of
in situ growing of the fermentation microorganisms in the fermentation vessel by means of the sugars added to the fermentation vessel, so as to maintain a steady-state population of fermentation microorganisms in the fermentation vessel, whereby further addition of microorganisms is not required, and wherein
the RSI setpoint corresponds to a steady state condition in the fermentation vessel.

3. The method of claim 1, wherein the fermentation process is a fed-batch fermentation process comprising at least one batch phase, feed phase and end phase, respectively, and having continuous addition of fermentation media during the at least one feed phase.

4. The method of claim 3, wherein
the step of determining an RSI setpoint is based on initial RSI measurements during the batch phase; and
the step of automatically adapting comprises automatically adapting, during the feed phase, the amount of sugar added to the fermentation vessel in a predetermined manner in response to RSI measurements during the feed phase and said RSI setpoint, so as to achieve and maintain said RSI setpoint.

5. The method of claim 4, wherein there is substantially no addition of microorganisms during the feed phase.

6. The method of claim 3, wherein the fermentation process is a repeated fed-batch fermentation process with continuous addition of fermentation media during at least two feed phases.

7. The method of claim 3, wherein the respective feed phase or feed phases of the fermentation process are extended so as to comprise a substantial portion of the fed-batch cycle.

8. The method of claim 1, further comprising the step of
secondary fermentation, in at least one secondary fermentation vessel arranged downstream of the fermentation vessel, to further increase the co-fermentation of sugars to fermentation products.

9. The method of claim 1, wherein the RSI setpoint comprises a target interval of the RSI or a target interval of the RSI rate of change, and the step of automatically adapting in turn comprises the steps of:
comparing measured, and preferably processed, values of the residual sugar indicator parameter to the RSI setpoint; and
adjusting the amount of sugar added to the fermentation vessel in response to the comparison, so as to reach and stay within the RSI setpoint.

10. The method of claim 1, wherein the measuring step involves density measurements.

11. The method of claim 1, wherein the measuring step involves refractive index (RI) measurements and the residual sugar indicator parameter RSI comprises a refractive index (RI) parameter.

12. The method of claim 1, wherein the measuring step involves a combination of measurements selected from the group of: optical measurements within UV, visual or IR wavelengths; measurements of carbon dioxide (CO₂) generation; and direct measurements of sugar concentration.

13. The method of claim 1, wherein the at least two different sugars are selected from the group of: glucose, mannose, xylose, arabinose, galactose, sucrose and fructose.

14. The method of claim 1, comprising
addition, to the fermentation vessel, of at least two separate sugar streams associated with the respective different sugars; and
individual adjustment of the respective at least two separate sugar streams associated with the respective different sugars.

15. A system for control of a microbial fermentation process involving co-fermentation, in a fermentation vessel, of sugars from lignocellulosic biomass to fermentation products by means of fermentation microorganisms, said system comprising:
means for continuous or semi-continuous addition, to the fermentation vessel, of a fermentation media comprising at least two different sugars;
measuring means for online measuring of a residual sugar indicator parameter RSI, which parameter directly or indirectly indicates the concentration of residual sugars, during fermentation in the fermentation vessel;
means for determining and setting an RSI setpoint based on a rate of change of the measured residual sugar indicator parameter, such that the RSI setpoint corresponds to a maximum rate of change; and
control means for automatically adapting the amount of sugar added to the fermentation vessel in a predetermined manner in response to the measured residual sugar indicator parameter RSI and the RSI setpoint, so as to achieve and maintain the RSI setpoint, whereby efficient co-fermentation of sugars to fermentation products is obtained.

16. The system of claim 15, further comprising
a secondary fermentation unit arranged downstream of the fermentation vessel, for further increased co-fermentation of sugars to fermentation products, and comprising at least two secondary fermentation vessels connected in series.

17. The system of claim 15, further comprising
a secondary fermentation unit arranged downstream of the fermentation vessel, for further increased co-fermentation of sugars to fermentation products, and comprising at least two secondary fermentation vessels connected in parallel.

18. The system of claim 15, comprising refractive index (RI) measurement means.

19. A system for producing fermentation products from lignocellulosic biomass, comprising the system for control of microbial fermentation of claim 15.
